(19) United States
(12) Patent Application Publication
(10) Pub. No.: US 2002/0197182 A1
(43) Pub. Date: Dec. 26, 2002

(54) Method and apparatus for directing ultrasonic energy

(75) Inventor: Bruce E. Minter, Eagle, ID (US)

Correspondence Address: Joseph W. Holland, P. O. Box 1840, Boise, ID, 83701-1840, US

(73) Assignee: Ozone Generator

(21) Appl. No.: 10/176,333

(22) Filed: Jun. 19, 2002

Related U.S. Application Data
(60) Provisional application No. 60/300,355, filed on Jun. 22, 2001.

(51) Int. Cl. A61L 2/00; A62B 7/08; A61L 9/00

(52) U.S. Cl. 422/20; 422/126; 422/305; 422/306

(57) ABSTRACT

An apparatus and method for transmitting ultrasonic energy. In one embodiment, the apparatus can include a vessel, such as a conduit, having a first end, a second end, and a vessel axis between the first and second ends. An ultrasonic energy emitter can be positioned toward the first end of the vessel to direct ultrasonic energy into the flowable substance during operation. An ultrasonic energy focuser can be positioned toward the first end of the vessel at least proximate to the ultrasonic energy emitter to focus the ultrasonic energy toward the vessel axis as the ultrasonic energy approaches the second end of the vessel. A reflector can be positioned toward the second end of the vessel to reflect the ultrasonic energy back toward the ult1.asonic energy emitter. A signal reverser can redirect at least part of the ultrasonic energy propagating away from the ultrasonic energy emitter.

### RELATED APPLICATIONS

This application claims the benefit of U.S. Provisional Application No. 60/300,355 filed Jun. 22, 2001. This application is also related to the following application assigned to a common assignee (a) “Ozone Generator”, Application Ser. No. 10/123,759 filed Apr. 15, 2002; and the following applications filed concurrently herewith (b) Method and Apparatus for Treating Fluid Mixtures with Ultrasonic Energy; (c); Method and Apparatus for Treating Fluid Mixtures with Ultrasonic Energy (d) and Method and Apparatus for Directing Ultrasonic Energy, which are all herein incorporated by reference.

### BACKGROUND

1. Technical Field

The present invention relates to methods and apparatuses for directing ultrasonic energy, for example, to treat mixtures such as agricultural or industrial waste streams.

2. Background

Many industrial, municipal and agricultural processes generate waste matter that is potentially harmful to the environment. Accordingly, a variety of processes have been developed to remove harmful elements from the waste matter before returning the waste matter to lakes, streams and oceans. Many conventional systems include filters, such as reverse osmosis filters that remove solid contaminants from the waste matter. However, because of environmental concerns, it may be difficult to dispose of the solid contaminants removed by the filters. Furthermore, the filters themselves must be periodically back-flushed, which can be a time consuming process. Accordingly, in one alternative process, microorganisms are disposed in the waste matter to consume or alter harmful elements in the waste matter. However, such systems generally process the waste matter in a batch mode and accordingly may be slow and labor intensive to operate. Another conventional approach is to sterilize waste matter streams with ultraviolet light. One problem with this approach is that the waste matter must be positioned very close to the light source, which can make ultraviolet systems slow, expensive and inefficient. Still another method includes exposing the waste matter stream to ozone, which can alter harmful elements in the waste matter stream. One problem with this approach is that the cost of generating effective quantities of ozone is generally so high that the process may not be economically feasible.

Yet another conventional approach is to expose the waste matter stream to ultrasonic energy. For example, one conventional method includes disposing the waste matter in a vessel and applying ultrasonic energy to the waste matter in a batch process. This conventional approach suffers from several drawbacks. For example, the batch process can be relatively slow. Furthermore, the efficiency with which ultrasonic energy is transmitted to the waste matter may be so low as to leave an unacceptable level of contaminants in the waste matter stream.

FIG. 1A is an isometric view of an ultrasonic energy emitter 10 in accordance with the prior art, configured to have an increased energy efficiency when compared with other devices. For example, the ultrasonic energy emitter 10 can have an electrically conductive signal reverser 11 attached to one surface with an adhesive 12, such as epoxy. A signal generator (not shown) transmits signals at a selected frequency via leads 13 and 14 connected to the emitter 10 and the signal reverser 11, respectively. The signal reverser 11 can reverse signals propagated by the ultrasonic energy emitter 10 in direction “X” so that the signals instead are directed in direction “Y.” Accordingly, the signal reverser 11 can direct more of the energy propagated by the emitter 10 in a single direction than can other ultrasonic emitter devices. Devices such as those shown in FIG. 1A are available from Morgan Matroc, Inc. of Bedford, Ohio.

FIG. 1B illustrates a partially conical ultrasonic emitter 10a attached to a generally cylindrical signal reverser 11a with an adhesive bond 12a and a threaded bolt (not shown) positioned in an aperture 15. Leads 13a and 14a supply signals at a selected frequency to the ult1.asonic energy emitter 10a. One drawback with the devices shown in FIGS. 1A and 1B is that the presence of the signal reversers can alter the frequency of the ultrasonic energy emitted by the ultrasonic energy emitters. Accordingly, it can be difficult to accurately control the characteristics of the ultrasonic energy emitted by these devices.

### SUMMARY

The present invention is directed toward methods and apparatuses for directing ultrasonic energy. An apparatus in accordance with one aspect of the invention can include a vessel having a first end, a second end opposite the first end, a vessel axis extending between the first and second ends, and a generally straight portion between the first and second ends. The vessel can be configured to removably contain a flowable substance and can include an ultrasonic energy emitter positioned toward the first end of the vessel to direct ultrasonic energy into the flowable substance during operation. The apparatus can further include an ultrasonic energy focuser positioned toward the first end of the vessel at least proximate to the ultrasonic energy emitter. The focuser can have a focusing surface configured to focus the ultrasonic energy toward the vessel axis as the ultrasonic energy moves toward the second end of the vessel. The focusing surface can include a first portion having a first parabolic shape with a first curvature and a second portion having a second parabolic shape with a second curvature different than the first curvature.

In another aspect of the invention, the apparatus can include an ultrasonic reflector positioned toward the second end of the vessel. The reflector can have a shaped, reflective surface positioned to reflect the ultrasonic energy toward the first end of the vessel. The reflective surface can be curved with an edge at least approximately tangent to a sidewall of the vessel and a tip on, and at least approximately tangent to, an axis spaced apart from the vessel sidewall and extending between the first and second ends of the vessel.

In still a further aspect of the invention, the ultrasonic energy emitter can include a first surface facing toward an interior of the vessel and a second surface facing opposite the first surface. The apparatus can further include a signal reverser positioned adjacent to the second surface of the ultrasonic energy emitter. The signal reverser can be biased against, but not adhered to, the ultrasonic energy emitter. The signal reverser can be positioned to receive a portion of ultrasonic energy emitted from the emitter and reflect at least part of the portion of ultrasonic energy into the flowable substance during operation. In yet a further aspect of the invention, the signal reverser can have a third surface adjacent to the second surface of the emitter, a fourth surface opposite the third surface, and a dimension between the third and fourth surfaces of approximately one quarter the wavelength of ultrasonic energy passing into the signal reverser.

The invention is also directed toward a method for focusing ultrasonic energy in a volume of flowable substance. The method can include directing the ultrasonic energy from an ultrasonic energy emitter into the flowable substance, impinging the ultrasonic energy on a shaped focusing surface to converge the ultrasonic energy toward a focal point spaced apart from the ultrasonic energy emitter, and exposing a selected constituent of the flowable substance to the ultrasonic energy as it converges toward the focal point. In another aspect of the invention, the method can be directed toward a method for reflecting ultrasonic energy in a volume of flowable substance. Accordingly, the method can include directing the ultrasonic energy from the ultrasonic energy emitter through the volume of flowable substance, and impinging the ultrasonic energy on a shaped reflecting surface spaced apart from the ultrasonic emitter to direct the ultrasonic energy back toward the ultrasonic energy emitter. The method can further include exposing a selected constituent of the flowable substance to the ultrasonic energy as it passes from the ultrasonic energy emitter to the reflecting surface and from the reflecting surface back toward the ultrasonic energy emitter.

### BRIEF DESCRIPTION OF THE DRAWINGS

FIGS. 1A and 1B illustrate ultrasonic energy emitters and signal reversers in accordance with the prior art.

FIG. 2 is a partially schematic, isometric view of an apparatus having ultrasonic energy focusers and ultrasonic energy reflectors in accordance with an embodiment of the invention.

FIG. 3 is a partially schematic, cross-sectional side elevational view of a portion of the apparatus shown in FIG. 2.

FIG. 4 is a cross-sectional side view of a portion of the apparatus shown in FIG. 2, including an ultrasonic energy focuser in accordance with an embodiment of the invention.

FIG. 5 is an isometric view of an ultrasonic energy reflector in accordance with an embodiment of the invention.

FIG. 6 is a cross-sectional, side elevational view of the ultrasonic energy reflector shown in FIG. 5 in accordance with an embodiment of the invention.

FIG. 7 is a partially schematic, isometric view of an apparatus having several processing vessels in accordance with another embodiment of the invention.

FIG. 8 is a partially schematic, cross-sectional view of a portion of an apparatus having two ultrasonic energy emitters in accordance with another embodiment of the invention.

FIG. 9 is a partially schematic, cross-sectional view of an apparatus for processing waste matter in a batch mode in accordance with another embodiment of the invention.

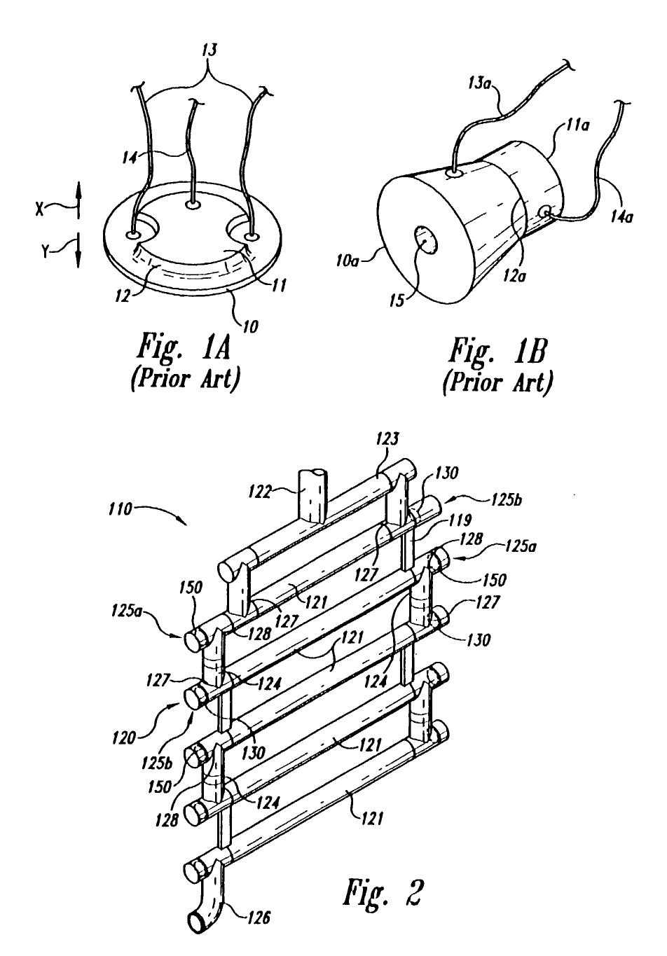

### DETAILED DESCRIPTION

The present disclosure describes apparatuses and methods for treating waste matter, such as aqueous waste matter streams. Many specific details of certain embodiments of the invention are set forth in the following description and in FIGS. 2-8 to provide a thorough understanding of these embodiments. One skilled in the art, however, will understand that the present invention may have several additional embodiments, or that the invention may be practiced without several of the details described below.

FIG. 2 is a partially schematic, isometric view of an apparatus 110 having ultrasonic energy sources 150 and ultrasonic energy reflectors 130 in accordance with an embodiment of the invention. In one aspect of this embodiment, the apparatus 110 can include a fluid-tight vessel 120 formed from a plurality of connected conduits or ducts. A waste matter stream can be introduced into the vessel 120 and exposed to ultrasonic energy emitted by the sources 150 and reflected by the reflectors 130 to reduce or eliminate potentially harmful characteristics of constituents.

In one embodiment, the vessel 120 can include an intake conduit 122 that receives the waste matter stream from a waste matter source, and an exit conduit 126 that can be coupled to downstream devices. The intake conduit 122 can be coupled to a manifold 123 that directs the waste matter stream into a plurality of elongated, serially connected channel or conduit portions 121. Each conduit portion 121 can include a first end 125a, a second end 125b, an entrance port 127 and an exit port 128. The length of each conduit portion 121 can be proportional to the power of the ultrasonic energy source 150 positioned in the conduit portion 121, and can be about 6 feet in one embodiment. The exit port 128 of each conduit portion 121 can be connected to the entrance port 127 of the next conduit portion 121 with a connecting conduit 124. Adjacent conduit portions 121 can be supported relative to each other with struts 119. The waste matter stream can proceed generally from the entrance port 127 of each conduit portion 121 to the exit port 128, then through the connecting conduit 124 to the entrance port 127 of the next conduit portion 121. The waste matter stream passes from the last conduit portion 121 into the exit conduit 126.

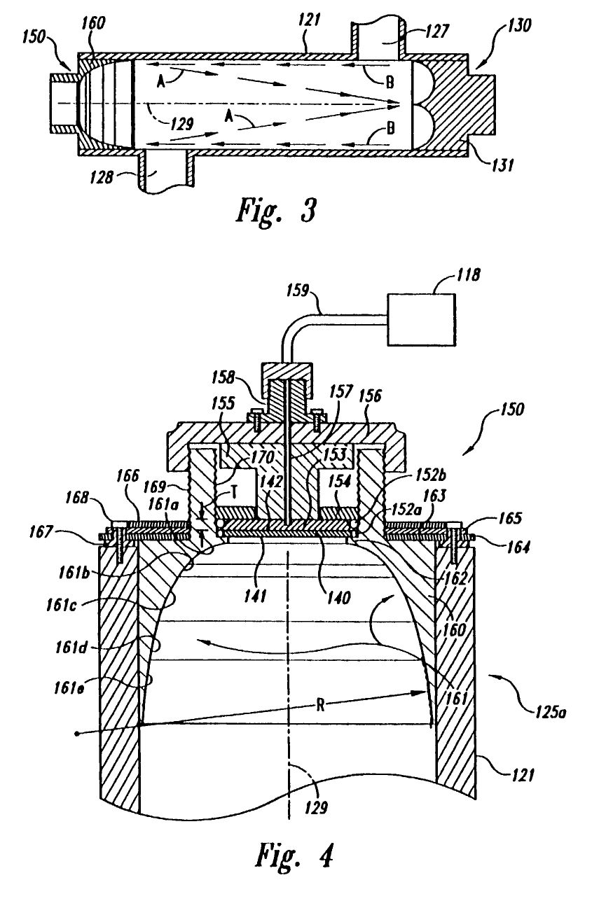

In one embodiment, each conduit portion 121 can include an ultrasonic energy source 150 (which can house a piezoelectric crystal or another ultrasonic energy emitter or generator), and an ultrasonic energy reflector 130. In one aspect of this embodiment, the ultrasonic energy source 150 can be positioned toward the first end 125a of the conduit portion 121, proximate to the exit port 128, and the ultrasonic energy reflector 130 can be positioned toward the second end 125b of the conduit portion 121, proximate to the entrance. Accordingly, the waste matter stream can travel toward the ultrasonic energy sources 150 as it moves through each conduit portion 121 from the entrance port 127 to the exit port 128. Alternatively, the ultrasonic energy sources 150 can be positioned toward the second end 125b of each conduit portion 121 with the waste matter stream traveling away from the ultrasonic energy sources 150. In either embodiment, the ultrasonic energy reflector 130 can be positioned to reflect (a) at least a portion of the ultrasonic energy generated by the ultrasonic energy source 150 and/or (b) products produced by the ultrasonic energy (such as cavitation bubbles) back toward the ultrasonic energy source 150, as described in greater detail below with reference to FIG. 3.

FIG. 3 is a partially schematic, cross-sectional side elevational view of one of the conduit portions 121 described above with reference to FIG. 2. The waste matter stream enters the conduit portion 121 through the entrance port 127 and proceeds through the conduit portion 121 along a vessel axis 129 to the exit port 128 (i.e., from right to left in FIG. 3). The ultrasonic energy source 150 can include a focuser body 160 that focuses ultrasonic energy toward the vessel axis 129, as shown schematically in FIG. 3 by arrows “A”. The ultrasonic energy reflector 130 can include a reflector body 131 that receives the focused ultrasonic energy and reflects the ultrasonic energy back toward the ultrasonic energy source 150, as shown schematically in FIG. 3 by arrows “B”. In one aspect of this embodiment, the reflected energy can be disposed generally annularly and concentrically around the focused energy. In other embodiments, the relative positions of the reflected and focused energies can have other arrangements. In either embodiment, focusing and reflecting the ultrasonic energy can increase the efficiency with which the ultrasonic energy treats the waste matter stream passing through the conduit portion 121, as described in greater detail below.

FIG. 4 is a cross-sectional view of the first end 125a of the conduit portion 121 and the ultrasonic energy source 150 shown in FIG. 3 in accordance with an embodiment of the invention. In one aspect of this embodiment, the focuser body 160 of the source 150 can include an emitter support member 162, such as a flange, that supports an ultrasonic emitter 140, such as a piezoelectric crystal. The focuser body 160 can further include a generally concave focusing surface 161 positioned to receive and focus ultrasonic energy emitted from the ultrasonic emitter 140. Accordingly, the focusing surface 161 can be curved to focus the ultrasonic energy toward the vessel axis 129 and the ultrasonic energy reflector 130 (FIG. 3). In one embodiment, the focusing surface 161 can include five segments, shown in FIG. 4 as segments 161a-161e. In a further aspect of this embodiment, each segment 161a-161e can be defined by a portion of a parabola revolved about the vessel axis 129. Each successive segment 161a-161e can have an average slope or inclination angle relative to the vessel axis 129 that is less than the inclination angle of the preceding segment. Accordingly, the median radius of curvature at the midpoint of successive segments (indicated by arrow “R” for segment 161e) can increase from segment 161a to segment 161e. For example, in one embodiment, segment 161a can have a midpoint radius of about 0.75 inches, segment 161b can have a midpoint radius of about 1.7 inches, segment 161c can have a midpoint radius of about 2.0 inches, segment 161d can have a midpoint radius of about 5.0 inches, and segment 161e can have a midpoint radius of about 7.0 inches. The focuser body 160 can be positioned in a conduit portion 121 having a diameter of about 2.75 inches. In other embodiments, the segments 161a-161e can have other midpoint radiuses of curvature and/or the conduit portion 121 can have other diameters.

In one aspect of this embodiment, the junction between adjacent segments 161a-161e can be smoothed or blended to reduce the discontinuity in slope resulting from the change from one parabolic surface to another. Alternatively, the junction can be unsmoothed or unblended. In a further alternate embodiment, the focusing surface 161 can include more or fewer segments than are shown in FIG. 4. In still a further alternate embodiment, the focusing surface 161 can include straight segments or segments having curves defined by non-parabolic shapes, so long as the focusing surface 161 tends to focus the ultrasonic energy emanating from the emitter 140. The focusing surface 161 can focus the energy along the central vessel axis 129 in one embodiment or, alternatively along other vessel axes in other embodiments.

In one embodiment, the ultrasonic emitter 140 can have a first surface 141 facing toward the fluid in the conduit portion 121, and a second surface 142 facing opposite the first surface 141. The apparatus 110 can further include an electrically conductive signal reverser 153 having an engaging surface adjacent to the second surface 142 of the ultrasonic emitter 140. A first O-ring 152a is positioned around the ultrasonic emitter 140, and a second O-ring 152b is positioned on a peripheral flange of the signal reverser 153. A contact probe 157 engages the signal reverser 153 and is attached to a connector 158. The connector 158 can be coupled with a coaxial lead 159 to a signal generator 118 to provide electrical power to the signal reverser 153. The signal reverser 153 can then transmit the electrical power to the ultrasonic emitter 140 to activate the emitter 140.

In a further aspect of this embodiment, the ultrasonic energy source 150 can include a retainer ring 154 that threadedly engages internal threads 170 of the focuser body 160. Accordingly, the retainer ring 154 can be rotated to engage the second O-ring 152b, which can (a) bias the signal reverser 153 against the emitter 140 while (b) sealing the second O-ring 152b against the focuser body 160 to protect the electrical connection between the signal reverser 153 and the probe 157 from exposure to the liquid in the conduit portion 121. The ultrasonic energy source 150 can further include a plunger 155 that extends through an aperture in the center of the retainer ring 154 to contact the signal reverser 153. A cap 156 can threadedly engage external threads 169 of the focuser body 160 to bias the plunger 155 against the signal reverser 153. In one aspect of this embodiment, the plunger 155 can include plastic material (such as Delrin™) and in other embodiments, the plunger 155 can include other materials. In either embodiment, the plunger 155 can also bias the signal reverser 153 against the emitter 140.

In one aspect of an embodiment of the ultrasonic energy source 150 shown in FIG. 4, the ultrasonic emitter 140 and the signal reverser 153 can be configured to enhance the efficiency with which ultrasonic energy is transmitted to the fluid within the conduit portion 121, when compared with some conventional devices. For example, the signal reverser 153 can have a thickness “T” that corresponds to about ¼ of the wavelength of the ultrasonic energy transmitted from the second surface 142 of the ultrasonic emitter 140 into tile signal reverser 153. In one specific embodiment in which the signal reverser 153 includes copper and the ultrasonic emitter 140 is configured to emit ultrasonic energy at a frequency of approximately 980 kHz, the signal reverser 153 can have a thickness “T” of approximately 0.25 inches. When the signal reverser 153 includes stainless steel, the thickness “T” can be approximately 0.125 inches for an ultrasonic frequency of about 980 kHz. When the signal reverser 153 includes brass, the thickness “T” can be approximately 1.0 inch for an ultrasonic frequency of about 980 kHz. In other embodiments, the signal reverser 153 can have other dimensions, depending on the material of the signal reverser 153 and the frequency with which the ultrasonic emitter 140 emits ultrasonic energy. In any of these embodiments, the signal reverser 153 can have a thickness “T” that corresponds to approximately ¼ of the wavelength of the ultrasonic energy passing ti1fough the signal reverser 153 from the ultrasonic emitter 140. Accordingly, the signal reverser 153 can reflect energy propagating from the second surface 142 of the emitter 140 back through the emitter 140 and into the waste matter in the conduit portion 121.

In another aspect of this embodiment, the ultrasonic emitter 140 is not adhesively bonded to the signal reverser 153, unlike some conventional arrangements. Instead, the signal reverser 153 is biased against the ultrasonic emitter 140 by the retainer ring 154 and/or the plunger 155. For example, in one particular embodiment, both the retainer ring 154 and the cap 156 can be tightened with a torque of from about 10 ft.-lbs. to about 20 ft.-lbs. In other embodiments, tile signal reverser 153 can be biased against the ultrasonic emitter 140 under other torques. An advantage of these embodiments is that it can be easier to control the frequency with which the ultrasonic emitter 140 propagates energy into the interior of the conduit portion 121. It is believed that biasing the signal reverser 153 against the emitter 140 (rather than gluing or otherwise adhering the signal reverser 153 to the emitter 140) can reduce or eliminate the effect of the signal reverser 153 on the frequency of ultrasonic energy propagated by the emitter 140. Accordingly, the ultrasonic emitter 140 can emit ultrasonic energy at the same or nearly the same frequency as tile signal transmitted to it by the signal generator 118. It is believed that this effect is due to the ability of the emitter J 40 and the signal reverser 153 to vibrate with at least some degree of independence relative to each other.

In still a further aspect of this embodiment, the focuser body 160 can be attached directly to the first end 125a of the conduit portion 121. For example, the focuser body 160 can include a radially extending washer support surface 163 that engages a washer 164. A support plate 165 is positioned against tile washer 164 and both tile support plate 165 and the washer 164 can be clamped against the washer support surface 163 with a lock ring 166 that engages the external threads 169 of the focuser body 160. Mounting bolts 168 can pass through apertures in the washer 164 and the support plate 165 to secure the focuser body 160 to the first end 125a of the conduit portion 121. The apparatus 110 can further include isolation washers 167 between the washer 164 and the end of the conduit portion 121 to electrically isolate the focuser body 160 from the conduit 121. In other embodiments, the ultrasonic energy source 150 can include other arrangements for attaching the focuser body 160 to the conduit portion 121.

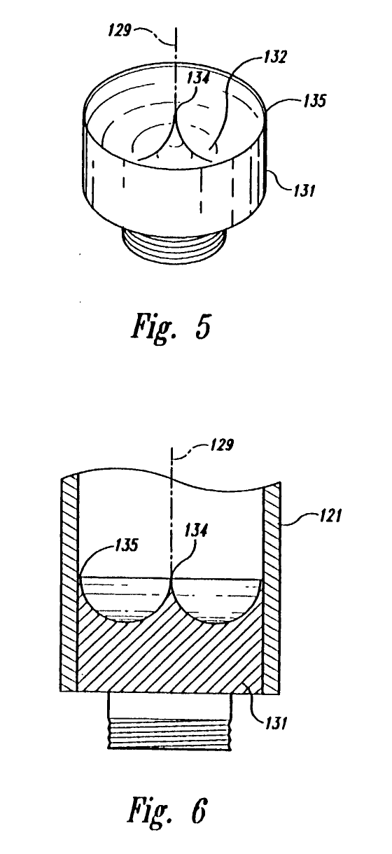

FIG. 5 is a side isometric view of tile reflector body 131 positioned opposite the ultrasonic energy source 150 (FIG. 3) in accordance with an embodiment of the invention. In one aspect of this embodiment, the reflector body 131 can include a generally concave, curved reflective surface 132 positioned to receive the ultrasonic energy propagating from the emitter 140 (FIG. 3), and reflect at least a portion of the ultrasonic energy away from the reflector body 131 and toward the emitter 140. In one aspect of this embodiment, the reflective surface 132 can be defined by a circular arc revolved about the vessel axis 129. Accordingly, the reflective surface 132 can have a tip or cusp portion 134 generally aligned with the vessel axis 129, and a rim portion 135 disposed radially outwardly from the tip portion 134. In other embodiments, the reflective surface 132 can have other shapes that receive the impinging ultrasonic energy and reflect the energy back into the waste matter stream. In any of these embodiments, the reflective surface 132 can be highly polished (for example, with a micro-finish or a mirror finish) to increase the efficiency with which the reflective surface 132 reflects ultrasonic energy.

FIG. 6 is a cross-sectional side view of the reflector body 131 shown in FIG. 5 positioned in a conduit portion 121 in accordance with an embodiment of the invention. As shown in FIG. 6, the rim portion 135 of the reflector body 131 can be at least approximately tangent the walls of the conduit portion 121. The tip portion 134 can be at least approximately tangent to the vessel axis 129 extending through the conduit portion 121. In a further aspect of this embodiment, the reflector body 131 can be secured to the conduit portion 121 with an arrangement of washers, support plates and mounting bolts (not shown in FIG. 6) generally similar to that described above with reference to FIG. 4. Alternatively, the reflector body 131 can be secured to the conduit portion 121 with other arrangements in other embodiments.

Operation of an embodiment of tile apparatus 110 is described below with reference to FIGS. 2 and 3. Referring first to FIG. 2, a liquid waste matter stream enters the apparatus 110 through the intake conduit 122, passes serially through each conduit portion 121, and exits the apparatus 110 through the exit conduit 126. Referring now to FIG. 3, the waste matter stream enters each conduit portion 121 through the entrance port 127 and flows toward the ultrasonic energy source 150 and the exit port 128. The ultrasonic energy source 150 generates ultrasonic energy and introduces the energy into the waste matter stream. The focuser 160 focuses the ultrasonic energy so that it converges toward the vessel axis 129 and the tip portion 134 of the reflector body 131. In one embodiment, the focuser 160 has a shape generally similar to that shown in FIG. 4, the conduit portion 121 has a length of approximately 6 feet and a diameter of approximately 2.75 inches, and the energy converges to a diameter of from about 0.25 inches to about 0.50 inches at the tip portion 134. The reflector 130 reflects the ult1.asonic energy back toward the ultrasonic energy source 150 with the reflected energy disposed generally annularly around the impinging energy.

During the operation of an apparatus 110 in accordance with an embodiment of the invention, the ultrasonic energy sources 150 can emit ultrasonic energy at a power and frequency that cause an aqueous (or other liquid) portion of the waste matter stream to cavitate. Accordingly, cavitation bubbles formed in the waste matter stream can grow in a cyclic fashion and ultimately collapse. This process creates very high temperatures, pressures, and thermal cycling rates. For example, it is estimated that this process can develop temperatures in the waste matter stream of up to 5,000 degrees Celsius, pressures of up to 1,000 atmospheres, and heating and cooling rates above 10 billion degrees Celsius per second for durations of less than 1 microsecond (see, for example, Suslick, “The Chemistry of Ultrasound,” in The Yearbook of Science and the Future, Encyclopedia Britannica, 138-145 (1994), incorporated herein in its entirety by reference). The temperatures and pressures developed by the collapsing cavitation bubbles can have several advantageous effects on tile constituents of the waste matter stream. For example, the collapsing bubbles can form radicals, such as OH radicals, which are unstable and can chemically interact with adjacent constituents in the waste matter stream to change the chemical composition of the adjacent constituents. In one such process, the OH radical can interact with nitrates in the waste matter stream to produce gases such as nitrogen dioxide. The following are sample steps in such a reaction:

1) NO₃+.OH .NO₃+OH

2) .NO₃+.OH HO₂. +.NO₂

3) .NO₂+.NO₂ .NO+.NO₃

4) .NO₂+.NO₂ .NO+.NO+O₂

5) .NO₂+.H .NO+.OH

6) .NO₂+.OH .NO+HO₂.

7) .NO₂+.O. .NO₂+O₂

In another embodiment, the reaction can continue (for example, in the presence of additional constituents) to produce nitrites. In yet another embodiment, the cavitating bubble can alter trichloroethylene, for example, in accordance with the following simplified reaction:

(Cl)₂C═CHCl+2H₂O . . . Cl₂+HCl+2H₂+2CO 1)

In other embodiments, the collapsing cavitation bubbles can have effects on other molecules that change a chemical composition of the molecules and/or change a phase of the molecules from a liquid or solid phase to a gaseous phase.

In still further embodiments, the collapsing cavitation bubbles can have other effects on other constituents of the waste matter stream. For example, the high pressures and temperatures generated by the collapsing cavitation bubbles can disrupt the molecular structure of the walls of living cells and can accordingly kill and break up pathogenic organisms, such as bacteria. Another effect of the collapsing cavitation bubbles can be to combust or oxidize constituents of tile waste matter stream. For example, the high temperature produced by the collapsing cavitation bubble can oxidize constituents of the waste matter stream, producing by-products such as carbon dioxide and ash. The carbon dioxide can evolve from the waste matter stream and the ash can be filtered from the waste matter stream. In still another embodiment, the collapsing cavitation bubbles can also separate constituents of the waste matter stream. For example, when the waste matter stream includes a mixture of oil, water, and an emulsifier, the collapsing cavitation bubbles can alter the molecular characteristics of the emulsifier and cause the emulsifier to lose its effectiveness. Accordingly, the oil and water can separate from each other and one or the other can be removed from the stream.

The collapsing cavitation bubbles can have other effects on the waste matter stream that alter the characteristics of the constituents of the stream in a manner that makes the constituents more benign and/or allows the constituents to be more easily removed from the waste matter stream. In any of these embodiments, several characteristics of the apparatus 110 can be selected to have desired effects on the waste matter stream. For example, the frequency of the ultrasonic energy transmitted by the ultrasonic energy sources 150 into the waste matter stream can be selected based on the resonant frequencies of constituents in the waste matter stream. In one particular embodiment, the frequency of the ultrasonic energy source 150 can be selected to be at or above a natural resonant frequency of molecules of constituents in the stream. In one further specific example, when the flow includes farm animal fecal waste in an aqueous solution, along with pathogens such as E. coli, the ultrasonic energy sources 150 can be selected to produce a distribution of ultrasonic waves having an energy peak at approximately 980 kilohertz. In other embodiments, the peak energy of the ultrasonic energy sources 150 can be selected to occur at other frequencies, depending for example on the types, relative quantities, and/or relative potential harmful effects of constituents in the stream. Accordingly, individual ultrasonic energy sources 150 can be selected to have a pal1icular, and potentially unique, effect on selected constituents of the waste matter stream.

In another embodiment, adjacent ultrasonic energy sources within the apparatus 110 can produce different frequencies. For example, the ultrasonic energy source 150 in the uppermost conduit portion 121 of FIG. 2 can emit energy at a higher frequency than that emitted by the energy source 150 in the next downstream conduit portion 121. An advantage of this arrangement for waste matter streams having multiple constituents (each of which is best affected by ultrasonic energy at a different frequency) is that the waste matter streams can be subjected to a plurality of frequencies, with each frequency tailored to affect a particular constituent of the waste matter stream. Such an arrangement can be more effective than some conventional arrangements for removing constituents from the waste matter stream in a single apparatus.

The geometry of the apparatus 110 can be selected to define the time during which any given constituent of the waste matter stream is subjected to the energy emitted by the ultrasonic energy sources 150. For example, the overall length of the flow path through the apparatus 10 and the rate at which the waste matter stream passes through the apparatus 110 can be selected according to the amount of suspended solids in the waste matter stream, with the overall residence time within the apparatus 110 being lower for waste matter streams having relatively few suspended solids and higher for waste matter streams having more suspended solids.

One feature of an embodiment of the apparatus 110 described above with reference to FIGS. 2-6 is that the focuser 160 and the reflector 130 can operate together to redirect energy within the conduit portion 121. For example, the focuser 160 can focus energy toward the reflector 130, and the reflector 130 can reflect the energy to travel generally parallel to the walls of the conduit por1ion 121 back toward the focuser 160. An advantage of this feature is that ultrasonic energy that would otherwise be absorbed by the end walls or the side walls of the conduit portion 121 is instead reintroduced into the flow passing through the conduit portion 121 to increase the likelihood for altering the constituents of the flow. For example, the degree to which bubbles form in the conduit portion 121 has been observed to be greater with the presence of the focuser 160 and the reflector 130 than without these components, with at least some of the bubbles tending to rise in the conduit pol1ion 121 when subjected to reflected ultrasonic energy.

Another feature of an embodiment of the apparatus 110 described above with reference to FIGS. 2-6 is that the signal reverser 153 is not adhesively bonded to the ultrasonic emitter 140 and is instead biased against the ultrasonic emitter 140. An advantage of this arrangement is that the signal reverser 153 can be less likely to alter the frequency of signals emanating from the ultrasonic emitter 140. Another advantage is that the ultrasonic emitter 140 can be less likely to overheat than an emitter that is bonded to a signal reflector. Accordingly, an arrangement of the emitter 140 and signal reverser 153 in accordance with an embodiment of the invention can have a longer life expectancy than conventional arrangements. Yet another feature of an embodiment of the apparatus 110 described above with reference to FIGS. 2-6 is that the signal reverser 153 can have a dimension generally normal to an emitting surface of tile emitter 140 that corresponds to approximately ¼ of the wavelength of ultrasonic energy passing into the signal reverser 153. Accordingly, the signal reverser 153 can more effectively redirect into the waste matter stream a portion of the ultrasonic energy that would otherwise propagate away from the waste matter stream.

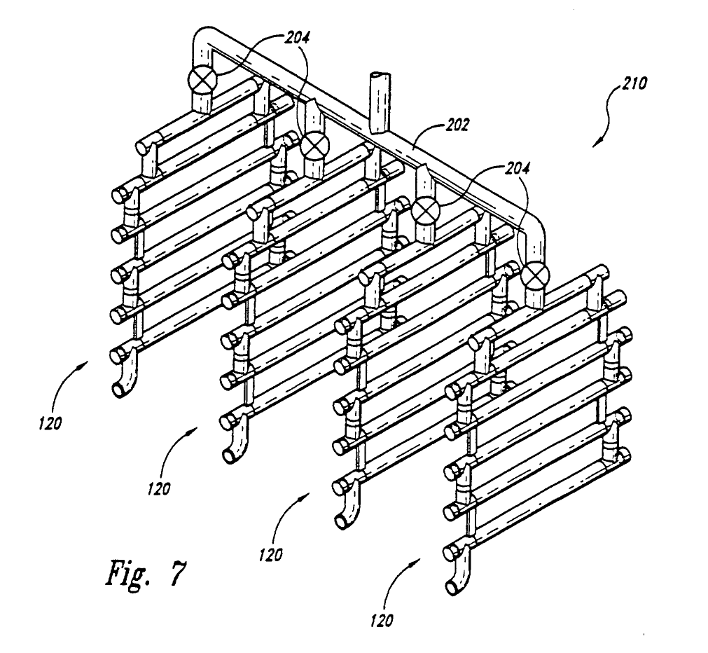

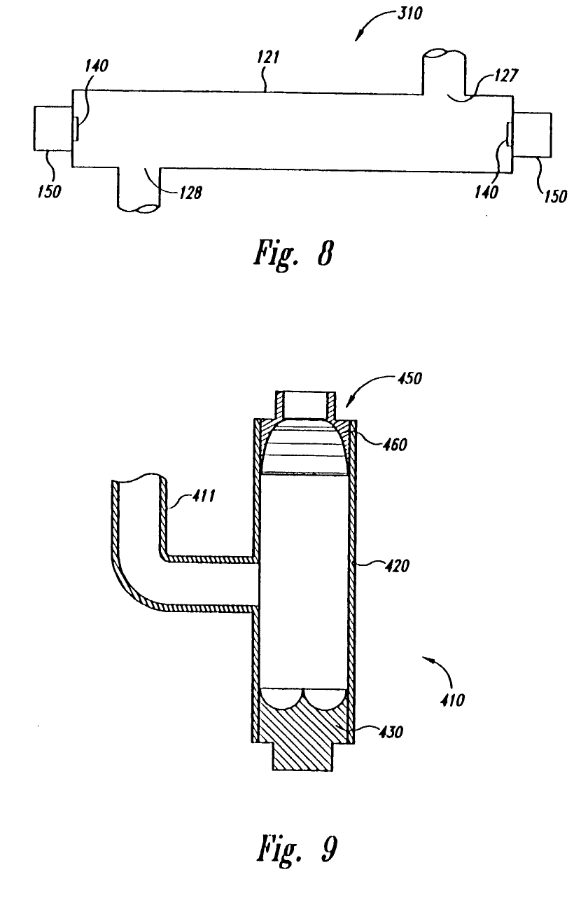

FIG. 7 is a partially schematic, isometric view of an apparatus 210 having a plurality of processing vessels 120 in accordance with another embodiment of the invention. In one aspect of this embodiment, the vessels 120 can be coupled to a common supply manifold 202. In a further aspect of this embodiment, each vessel 120 can include a selector valve 204 at a junction with the supply manifold 202. Accordingly, incoming waste matter can be selectively directed into one or more of the vessels 120. In a further aspect of this embodiment, each vessel 120 can be configured to process a particular type of waste matter stream (for example, by including ultrasonic energy sources tuned to a particular ultrasonic frequency). Accordingly, the incoming waste matter stream can be selectively directed to that vessel 120 configured to best interact with the constituents of that waste matter stream. In other embodiments, the apparatus 210 can have other arrangements. FIG. 8 is a partially schematic, cross-sectional side elevational view of a portion of an apparatus 310 that includes a conduit portion 121 having an entrance port 127 and an exit port 128 arranged in a manner generally similar to that of the apparatus 110 described above with reference to FIG. 2. In one aspect of this embodiment, the apparatus 310 can include two ultrasonic energy sources 150 positioned at opposite ends of the conduit portion 121. Each ultrasonic energy source 150 can have an ultrasonic energy emitter 140 generally similar to those described above with reference to FIGS. 2-7. Accordingly, the apparatus 310 can increase the amount of ultrasonic energy introduced to the waste matter stream passing through the conduit portion 121 compared with conventional devices having a single ultrasonic energy source. Conversely, an advantage of a device having an ultrasonic focuser and reflector generally similar to those described above with reference to FIGS. 2-6 is that the reflected ultrasonic energy can be directed around the energy emanating from the ultrasonic emitter 140 to impinge on the focuser 160, rather than directly on the ultrasonic emitter 140. Accordingly, the ultrasonic emitter 140 may be less subject to long-term wear than the ultrasonic energy sources 150 shown in FIG. 8.

FIG. 9 is a partially schematic, cross-sectional view of an apparatus 410 configured to process waste matter in a batch mode in accordance with another embodiment of the invention. In one aspect of this embodiment, the apparatus 410 can include a vessel 420 having an ultrasonic energy source 450 and a focuser 460 at one end, and an ultrasonic energy reflector 430 at the opposite end. The waste matter stream can be introduced to the vessel through an entrance/exit port 411 and subjected to ultrasonic energy in a manner generally similar to that described above with reference to FIGS. 2-6. After a selected period of time, the waste matter stream can be removed through the entrance/exit port 411. In an alternative arrangement, the apparatus 410 can include two ultrasonic energy sources 450, one at each end of the vessel 420, in a manner generally similar to that described above with reference to FIG. 8.

From the foregoing, it will be appreciated that specific embodiments of the invention have been described herein for purposes of illustration, but that various modifications may be made without deviating from the spirit and scope of the invention. For example, in one alternate embodiment, the apparatus can include a support member that supports the ultrasonic energy emitter, but does not have a focusing surface. The apparatus can include a reflector and/or a signal reverser arranged in a manner generally similar to one or more of the embodiments described above with reference to FIGS. 2-9. Accordingly, the invention is not limited except as by the appended claims.

I claim:

1. An apparatus for focusing ultrasonic energy, comprising:
a vessel having a first end, a second end opposite the first end, a vessel axis extending between the first and second ends, and a generally straight portion between the first and second ends, the vessel being configured to removably contain a flowable substance;
an ultrasonic energy emitter positioned toward the first end of the vessel to direct ultrasonic energy into the flowable substance during operation; and
an ultrasonic energy focuser positioned toward the first end of the vessel at least proximate to the ultrasonic energy emitter, the focuser having a focusing surface configured to focus the ultrasonic energy toward the vessel axis as the ultrasonic energy moves toward the second end of the vessel.

2. The apparatus of claim 1 wherein a first portion of the focusing surface has a first generally parabolic shape with a first curvature and a second portion of the reflective surface has a second parabolic shape with a second curvature different than the first curvature, the second portion being non-tangential to the first portion at an intersection between the first and second portions.

3. The apparatus of claim 1 wherein the vessel includes a cylinder generally axisymmetric about the vessel axis, further wherein the emitter is intersected by the vessel axis and the focusing surface extends radially outwardly from the emitter and axially away from the emitter, the focusing surface having first, second, third, fourth and fifth curved portions with each curved portion defining an annular region about the vessel axis and having an average slope relative to the vessel axis, the first, second, third, fourth and fifth portions being positioned successively further from the emitter and having successively smaller average slopes relative to the vessel axis.

4. The apparatus of claim 1 wherein the vessel includes a cylinder generally axisymmetric about the vessel axis, further wherein the emitter is intersected by the vessel axis and the focusing surface extends radially outwardly from the emitter and axially away from the emitter, the focusing surface having first, second, third, fourth and fifth curved portions with each curved portion defining an annular region about the vessel axis and having a median radius of curvature perpendicular to the vessel axis, the first, second, third, fourth and fifth portions having successively larger median radiuses of curvature.

5. The apparatus of claim 1 wherein the vessel includes a channel elongated along the vessel axis and axisymmetlic about the vessel axis.

6. The apparatus of claim 1 wherein the ultrasonic emitter has a central portion intersected by the vessel axis and further wherein the focusing surface is disposed radially outwardly from the vessel axis.

7. The apparatus of claim 1 wherein the emitter is sized to emit ultrasonic energy at a selected power and the vessel has a vessel length between the first and second ends, the vessel length being directly proportional to the selected power.

8. The apparatus of claim 1 wherein the ultrasonic emitter has a first surface facing toward an interior of the vessel and a second surface facing opposite the first surface, and wherein the apparatus further comprises a signal reverser positioned adjacent to the second surface of the ultrasonic energy emitter, the signal reverser being biased against but not adhered to the ultrasonic energy emitter, the signal reverser being positioned to receive a portion of ultrasonic energy emitted from the emitter and direct at least part of the portion of ultrasonic energy into the flowable substance during operation.

9. The apparatus of claim 1 wherein the ultrasonic energy emitter includes an electrically powered piezoelectric transducer.

10. The apparatus of claim 1 wherein the ultrasonic energy emitter is removably coupled to the focuser and the focuser is removably coupled to the vessel.

11. The apparatus of claim 1 wherein the ultrasonic energy emitter is configured to emit ultrasonic energy having a peak power at a frequency of approximately 980 kilohertz.

12. The apparatus of claim 1 wherein the ultrasonic emitter has a first surface facing toward the second end of the vessel and a second surface facing away from the first surface, the first surface being intersected by the vessel axis, the ultrasonic emitter being configured to emit ultrasonic energy at a power and frequency sufficient to change a phase and/or a chemical composition of a selected constituent in the flowable substance.

13. The apparatus of claim 1 wherein the vessel includes an inlet port proximate to the second end and an exit port proximate to the first end.

14. The apparatus of claim 1 wherein the vessel has a port positioned to receive an incoming flow of the flowable substance and pass an outgoing flow of the flowable substance during operation.

15. An apparatus for reflecting ultrasonic energy, comprising:
a vessel having a first end, a second end opposite the first end, and a generally straight portion between the first and second ends, the vessel being configured to removably contain a flowable substance;
an ultrasonic energy emitter positioned toward the first end of the vessel to transmit ultrasonic energy into the vessel; and
an ultrasonic reflector positioned toward the second end of the vessel, the reflector having a shaped, reflective surface positioned to reflect the ultrasonic energy toward the first end of the vessel.

16. The apparatus of claim 15 wherein the reflective surface has a shape defined generally by an arc revolved about a vessel axis extending between the first and second ends of the vessel.

17. The apparatus of claim 15 wherein the vessel has a sidewall and an axis extending between the first and second ends of the vessel, the axis being spaced apart from and generally parallel to the sidewall, and wherein the reflective surface is curved and has an edge at least approximately tangent to the sidewall, the reflective surface further having a tip at least approximately tangent to the axis.

18. The apparatus of claim 15 wherein the reflective surface has a micro finish and/or a mirror finish.

19. The apparatus of claim 15 wherein the vessel has a circular cross-sectional shape.

20. The apparatus of claim 15 wherein the vessel has a vessel axis extending between the first and second ends of the vessel and wherein the vessel has a vessel sidewall generally parallel to the vessel axis, further wherein the reflector is positioned to reflect the ultrasonic energy along an axis generally parallel to the vessel axis and the sidewall.

21. The apparatus of claim 15 wherein the vessel is elongated between the first and second ends along a generally straight vessel axis and further wherein the vessel, the emitter and the reflector have shapes that are axisymmetric about the vessel axis.

22. The apparatus of claim 15 wherein at least a portion of the reflective surface has a circular cross-sectional shape when intersected by a plane passing through an axis extending from the first end of the vessel to the second end of the vessel.

23. The apparatus of claim 15, further comprising a focusing surface positioned proximate to the ultrasonic energy emitter and configured to focus toward the ultrasonic reflector ultrasonic energy emanating from the ultrasonic energy emitter.

24. The apparatus of claim 23 wherein a first portion of the focusing surface has a first parabolic shape with a first curvature and a second portion of the focusing surface has a second parabolic shape with a second curvature different than the first curvature.

25. The apparatus of claim 15 wherein the vessel includes a fluid inlet port toward the second end between the reflector and the emitter, and a fluid outlet port toward the first end between the inlet poll and the emitter.

26. The apparatus of claim 15 wherein the vessel includes a first channel and wherein the apparatus further comprises:
a second channel; and
an inlet manifold coupled to the first and second channels to direct a first portion of the flowable substance into the first channel and a second portion of the flowable substance into the second channel.

27. The apparatus of claim 15 wherein the vessel includes a first channel, the ultrasonic emitter is a first ultrasonic emitter, and wherein the apparatus further comprises:
a second channel;
an inlet manifold coupled to the first and second channels to direct a first portion of the flowable substance into the first channel and a second portion of the flowable substance into the second channel; and
a second ultrasonic emitter positioned in the second channel, with the first ultrasonic emitter configured to emit ultrasonic energy at a first frequency and the second ultrasonic emitter configured to emit ultrasonic energy at a second frequency different than the first frequency.

28. The apparatus of claim 15 wherein the ultrasonic energy emitter is a first ultrasonic energy emitter and the vessel includes a first conduit portion housing the first ultrasonic energy emitter, and wherein the vessel further includes a second conduit portion downstream from the first conduit portion and housing a second ultrasonic energy emitter.

29. The apparatus of claim 15 wherein the vessel has a port positioned to receive an incoming flow of the flowable substance and pass an outgoing flow of the flowable substance during operation.

30. The apparatus of claim 15 wherein the vessel includes an entrance port positioned to receive an incoming flow of the flowable substance and an exit port positioned to pass an outgoing flow of the flowable substance during operation.

31. The apparatus of claim 15 wherein the ultrasonic energy emitter is configured to emit ultrasonic energy having a peak power at a frequency of approximately 980 kilohertz.

32. The apparatus of claim 15 wherein the ultrasonic emitter has a first surface facing toward the second end and a second surface facing away from the first surface, the first surface being intersected by a vessel axis extending between the rust and second ends of the vessel, the ultrasonic emitter being configured to emit ultrasonic energy at a power and frequency sufficient to change a phase and/or a chemical composition of a selected constituent in the flowable substance.

33. An apparatus for focusing ultrasonic energy, comprising:
a vessel having a first end, a second end opposite the first end, and a generally straight portion between the first and second ends, the vessel being configured to removably contain a flowable substance; and
a first ultrasonic energy emitter positioned toward the first end of the vessel to direct first ultrasonic energy into the vessel and toward the second end of the vessel; and
a second ultrasonic energy emitter positioned toward the second end of the vessel to direct second ultrasonic energy into the vessel and toward the first end of the vessel.

34. The apparatus of claim 33 wherein the first ultrasonic energy emitter is configured to emit ultrasonic energy having a peak energy level at a first frequency and the second ultrasonic energy emitter is configured to emit ultrasonic energy having a peak energy level at a second frequency different than the first frequency.

35. The apparatus of claim 33 wherein the first ultrasonic energy emitter is configured to emit ultrasonic energy at a selected power and a length of the vessel between the first and second ultrasonic energy emitters is directly proportional to the selected power.

36. An apparatus for focusing and reflecting ultrasonic energy, comprising:
a vessel having a first end, a second end opposite the first end, a vessel axis extending between the rust and second ends, and a generally straight portion between the first and second ends, the vessel being configured to removably contain a flowable substance;
an ultrasonic energy emitter positioned toward the first end of the vessel to direct ultrasonic energy into the vessel;
an ultrasonic focuser positioned toward the first end of the vessel proximate to the ultrasonic energy emitter, the focuser having a focusing surface positioned to focus the ultrasonic energy toward the vessel axis as the ultrasonic energy approaches the second end of the vessel; and
an ultrasonic reflector positioned toward the second end of the vessel, the reflector having a reflective surface positioned to receive the ultrasonic energy from the emitter and the focuser and reflect the ultrasonic energy toward the first end of the vessel.

37. The apparatus of claim 36 wherein the focuser is positioned to focus the ultrasonic energy at a focal point, and wherein at least a portion of the reflective surface is positioned at the local point, the reflective surface having a shape defined by a section of a circle revolved about an axis extending between the first and second ends of the vessel, the reflective surface further having a tip positioned approximately at the focal point.

38. The apparatus of claim 36 wherein the focuser is positioned to direct a converging beam of ultrasonic energy toward the second end of the vessel and the reflector is positioned to direct reflected ultrasonic energy toward the first end of the vessel with at least a portion of the reflected ultrasonic energy disposed annularly about the converging beam.

39. The apparatus of claim 36 wherein the focuser is positioned to focus the ultrasonic energy at a focal point, and further wherein at least a portion of the reflective surface is positioned at the focal point.

40. The apparatus of claim 36 wherein the ultrasonic energy emitter is sized to emit ultrasonic energy at a selected power and wherein a length of the vessel between the ultrasonic emitter and the reflector is selected to be directly proportional to the selected power.

41. An apparatus for transmitting ultrasonic energy, comprising:
a support member;
an ultrasonic energy emitter engaged with the support member, the ultrasonic energy emitter having a first surface and a second surface facing opposite the first surface; and
signal reverser positioned adjacent to the second surface of the ultrasonic energy emitter, the signal reverser being biased against but not adhered to the ultrasonic energy emitter, the signal reverser being positioned to receive a portion of ultrasonic energy emanating from the emitter and direct at least part of the portion of ultrasonic energy back into and through the emitter.

42. The apparatus of claim 41, further comprising a vessel having a first end and a second end opposite the first end, the vessel being configured to removably contain a flowable substance, the support member being coupled to the vessel and engaged with the ultrasonic energy emitter to support the emitter relative to the vessel.

43. The apparatus of claim 42 wherein the vessel includes an entrance port proximate to the second end, the vessel further including an exit port proximate to the first end.

44. The apparatus of claim 42 wherein the vessel includes a vessel axis extending between the first and second ends, and wherein the apparatus further comprises:
an ultrasonic energy focuser positioned toward the first end of the vessel at least proximate to the ultrasonic energy emitter, the focuser having a focusing surface configured to focus the ultrasonic energy toward the vessel axis when the ultrasonic energy approaches the second end of the vessel; and
an ultrasonic energy reflector positioned toward the second end of the vessel and having a shaped reflective surface configured to reflect the ultrasonic energy back toward the ultrasonic energy emitter.

45. The apparatus of claim 41, further comprising a biasing member threadedly engaged with the support member and coupled to the signal reverser to bias the signal reverser into engagement with the ultrasonic energy emitter.

46. The apparatus of claim 41 wherein the ultrasonic energy emitter includes a piezoelectric crystal.

47. The apparatus of claim 41 wherein the ultrasonic energy emitter is configured to emit ultrasonic energy at a selected frequency and wherein the signal reverser has a third surface adjacent to the second surface of the ultrasonic energy emitter, the signal reverser further having a fourth surface facing opposite the third surface, and further wherein the signal reverser has a dimension between the third and fourth surfaces that is approximately one quarter of a wavelength of the ultrasonic energy in the signal reverser.

48. An apparatus for transmitting ultrasonic energy, comprising:
a signal generator configured to transmit an electrical signal at a selected ultrasonic frequency;
an ultrasonic energy emitter operatively coupled to the signal generator to emit ultrasonic energy at least approximately at the selected frequency, the ultrasonic energy emitter having a first surface and a second surface facing opposite the first surface; and
a signal reverser operatively coupled between the signal generator and the ultrasonic energy emitter, the signal reverser having a third surface positioned adjacent to the second surface of the ultrasonic energy emitter to direct at least some of the ultrasonic energy emitted from the second surface of the ultrasonic energy emitter back through the ultrasonic energy emitter to propagate from the first surface of the ultrasonic emitter, the signal reverser further having a fourth surface facing opposite the third surface, the signal reverser still further having a dimension between the third and fourth surfaces that corresponds to about one quarter of a wavelength of the ultrasonic energy traveling in the signal reverser.

49. The apparatus of claim 48 wherein the selected frequency is approximately 980 kHz, the signal reverser includes copper, and the dimension between the third and fourth surfaces of the signal reverser is about 0.25 inches.

50. The apparatus of claim 48 wherein the selected frequency is approximately 980 kHz, the signal reverser includes stainless steel, and the dimension between the third and fourth surfaces of the signal reverser is about 0.125 inches.

51. The apparatus of claim 48 wherein the selected frequency is approximately 980 kHz, the signal reverser includes brass, and the dimension between the third and fourth surfaces of the signal reverser is about 1.0 inch.

52. The apparatus of claim 48 wherein the signal reverser is biased against but not adhered to the ultrasonic energy emitter.

53. An apparatus for focusing ultrasonic energy in a stream of a flowable substance, comprising:
a generally cylindrical conduit having a first end, a second end opposite the first end, an entrance port proximate to the second end, and an exit port proximate to the first end, the conduit further having a generally straight centerline axis extending between the first and second ends, the conduit further having a sidewall generally parallel to the centerline axis;
an ultrasonic energy emitter positioned toward the first end of the conduit and having a first surface facing toward the second end of the conduit, the ultrasonic energy emitter further having a second surface facing away from the first surface, the first surface being intersected by the centerline axis, the ultrasonic energy emitter being configured to emit ultrasonic energy at a power and frequency sufficient to change a phase and/or a chemical composition of a selected constituent in the flowable substance;
an ultrasonic focuser positioned toward the first end of the conduit proximate to the ultrasonic energy emitter, the focuser having a focusing surface positioned to focus the ultrasonic energy toward the centerline axis as the ultrasonic energy approaches the second end of the conduit, the focusing surface including at least a first and second portion with the first portion defining a first parabolic shape and the second portion defining a second parabolic shape different than the first parabolic shape; and
an ultrasonic reflector positioned toward the second end of the conduit, the reflector having a reflective surface positioned to reflect the ultrasonic energy toward the first end of the conduit, the reflective surface being defined by a section of a circle revolved about the centerline axis, the reflective surface having an edge adjacent to the sidewall and at least approximately tangent to the sidewall, the reflective surface further having a tip positioned on the centerline axis and at least approximately tangent to the centerline axis.

54. The apparatus of claim 53, further comprising a signal reverser positioned adjacent to the second surface of the ultrasonic energy emitter, the signal reverser being biased against but not adhered to the ultrasonic energy emitter, the signal reverser being positioned to receive a portion of ultrasonic energy emitted from the emitter and direct at least part of the portion of ultrasonic energy into the flowable substance during operation.

55. A method for focusing ultrasonic energy in a volume of a flowable substance, comprising:
directing the ultrasonic energy from an ultrasonic energy emitter into the flowable substance;
impinging the ultrasonic energy on a shaped focusing surface to converge the ultrasonic energy toward a focal point spaced apart from the ultrasonic energy emitter; and
exposing a selected constituent of the flowable substance to the ultrasonic energy as it converges toward the focal point.

56. The method of claim 55, further comprising changing a phase and/or a chemical composition of a selected constituent in the flowable substance.

57. The method of claim 55 wherein exposing the flowable substance to ultrasonic energy includes cavitating a liquid portion of the flowable substance to generate heat, and wherein the method further includes altering a chemical composition of a selected constituent in the flowable substance by oxidizing the selected constituent to produce an ash and a gas.

58. The method of claim 55 wherein exposing the flowable substance to the ultrasonic energy includes cavitating a liquid portion of the flowable substance to generate heat, and wherein the method further includes killing pathogens in the flowable substance by exposing the pathogens to the heat.

59. The method of claim 55 wherein exposing the flowable substance to ultrasonic energy includes cavitating a liquid portion of the flowable substance.

60. The method of claim 55 wherein impinging the ultrasonic energy includes impinging the ultrasonic energy on a first portion of the focusing surface having a first parabolic shape with a first curvature and impinging the ultrasonic energy on a second portion of the focusing surface having a second parabolic shape with a second curvature different than the first curvature.

61. The method of claim 55, further comprising directing the flowable substance into a conduit and directing the ultrasonic energy into the flowable substance while the flowable substance is in the conduit.

62. The method of claim 55 wherein the flowable substance is in a vessel having a generally circular cross-sectional shape and wherein directing the ultrasonic energy includes directing the ultrasonic energy radially outwardly from the ultrasonic energy emitter toward the focusing surface.

63. The method of claim 55 wherein directing the ultrasonic energy includes directing the ultrasonic energy with a peak power at a frequency of approximately 980 kilohertz.

64. The method of claim 55, further comprising receiving at least a portion of the focused ultrasonic energy at a reflective surface and reflecting at least part of the portion of ultrasonic energy back into the flowable substance.

65. A method for reflecting ultrasonic energy in a volume of a flowable substance, comprising:
directing the ultrasonic energy from an ultrasonic energy emitter through the volume of flowable substance;
impinging the ultrasonic energy on a shaped reflecting surface spaced apart from the ultrasonic energy emitter to direct the ultrasonic energy back toward the ultrasonic energy emitter; and
exposing a selected constituent of the flowable substance to the ultrasonic energy as it passes from the ultrasonic energy emitter to the reflecting surface and from the reflecting surface back toward the ultrasonic energy emitter.

66. The method of claim 65, further comprising changing a phase and/or a chemical composition of a selected constituent in the flowable substance.

67. The method of claim 65 wherein exposing the flowable substance to ultrasonic energy includes cavitating a liquid portion of the flowable substance to generate heat, and wherein the method further includes altering a chemical composition of a selected constituent in the flowable substance by oxidizing the selected constituent to produce an ash and a gas.

68. The method of claim 65 wherein exposing the flowable substance to ultrasonic energy includes cavitating a liquid portion of the flowable substance to generate heat, and wherein the method further includes killing pathogens in the flowable substance by exposing the pathogens to the heat.

69. The method of claim 65 wherein exposing the flowable substance to ultrasonic energy includes cavitating a liquid portion of the flowable substance.

70. The method of claim 65 wherein impinging the ultrasonic energy on a reflective surface includes impinging the ultrasonic energy on a reflective surface having a shape defined by a section of a circle revolved about an axis extending between the ultrasonic energy emitter and the reflecting surface.

71. The method of claim 65, further comprising focusing the ultrasonic energy in a region proximate to the ultrasonic energy emitter to converge the ultrasonic energy toward a focal point at least proximate to the reflective surface.

72. The method of claim 65, further comprising directing the flowable substance though an entrance poll of a vessel, exposing the flowable substance to the ultrasonic energy while the flowable substance is in the vessel, and removing the flowable substance from the vessel through an exit poll spaced apart from the entrance port.

73. The method of claim 65 wherein directing the ultrasonic energy includes directing the ultrasonic energy with a peak power at a frequency of approximately 980 kilohertz.

74. A method for focusing and reflecting ultrasonic energy in a volume of a flowable substance, comprising:
directing the ultrasonic energy from an ultrasonic energy emitter into the volume of flowable substance;
impinging the ultrasonic energy on a focusing surface to converge the ultrasonic energy toward a focal point spaced apart from the ultrasonic energy emitter;
impinging the ultrasonic energy on a reflecting surface spaced apart from the ultrasonic energy emitter to direct the ultrasonic energy back toward the ultrasonic energy emitter; and
exposing a selected constituent of the flowable substance to the ultrasonic energy as the ultrasonic energy converges toward the focal point and passes from the reflecting surface back toward the ultrasonic energy emitter.

75. The method of claim 74, further comprising focusing the ultrasonic energy toward a focal point and reflecting the ultrasonic energy from the focal point by impinging the ultrasonic energy on a reflective surface defined by a section of a circle revolved about an axis extending between the emitter and the reflective surface and having a tip at least proximate to the focal point.

76. The method of claim 74, further comprising:
directing a converging portion of ultrasonic energy toward the reflective surface; and
directing reflected ultrasonic energy toward the emitter with at least a portion of the reflected ultrasonic energy disposed annularly about the converging portion.

77. The method of claim 74 wherein impinging the ultrasonic energy on a reflecting surface includes impinging the ultrasonic energy on a surface having a shape defined by a section of a circle revolved about a vessel axis extending between the focusing and reflecting surfaces.

78. A method for assembling an ultrasonic energy source, comprising:
supporting an ultrasonic energy emitter, the emitter having a first surface and a second surface facing opposite the first surface;
engaging a signal reverser with the second surface of the ultrasonic energy emitter; and
biasing the signal reverser into engagement with the ultrasonic energy emitter without fixedly adhering the signal reverser to the ultrasonic energy emitter.

79. The method of claim 78 wherein supporting the ultrasonic energy emitter includes engaging the emitter with a support member coupled to a vessel configured to hold a flowable substance, and wherein biasing the signal reverser into engagement with the ultrasonic energy emitter includes threadedly attaching a biasing member to the support member to advance the biasing member into engagement with the emitter.

80. The method of claim 78, further comprising:
coupling a signal generator to the ultrasonic emitter;
transmitting a signal from the signal generator to the ultrasonic energy emitter at a selected frequency; and
selecting the signal reverser to have a third surface adjacent to the second surface of the ultrasonic energy emitter, a fourth surface opposite the third surface and a dimension between the third and fourth surfaces of approximately one quarter of a wavelength of the ultrasonic energy in the signal reverser at approximately the selected frequency.

81. A method for transmitting ultrasonic energy, comprising:
generating an electrical signal at a selected frequency;
coupling the signal to an ultrasonic energy emitter to emit ultrasonic energy at least approximately at the selected frequency; and
passing at least a portion of the ultrasonic energy into a signal reverser adjacent to the ultrasonic energy emitter, the signal reverser having a first surface adjacent to the ultrasonic energy emitter, a second surface facing opposite the first surface, and a dimension between the first and second surfaces of approximately one quarter of a wavelength of the ultrasonic energy in the signal reverser.

82. The method of claim 81, further comprising biasing the signal reverser against the ultrasonic energy emitter without fixedly adhering the signal reverser to the ultrasonic energy emitter.

83. The method of claim 81, further comprising:
selecting the selected frequency to be approximately 980 kHz;
selecting the signal reverser to include copper; and
selecting the dimension between the first and second surfaces of the signal reverser to be about 0.25 inches.

84. The method of claim 81, further comprising:
selecting the selected frequency to be approximately 980 kHz;
selecting the signal reverser to include stainless steel; and
selecting the dimension between the first and second surfaces of the signal reverser to be about 0.125 inches.

85. The method of claim 81, further comprising:
selecting the selected frequency to be approximately 980 kHz;
selecting the signal reverser to include brass; and
selecting the dimension between the first and second surfaces of the signal reverser to be about 1.0 inch.

86. A method for transmitting ultrasonic energy to a flowable substance, comprising:
directing the flowable substance into a vessel having a first end, a second end opposite the first end, and a generally straight portion between the first and second ends;
directing first ultrasonic energy into the flowable substance from a first ultrasonic energy emitter positioned toward the first end of the vessel; and
directing second ultrasonic energy into the flowable substance from a second ultrasonic energy emitter positioned toward the second end of the vessel.

87. The method of claim 86 wherein directing the first ultrasonic energy includes directing the first ultrasonic energy with a peak energy level at a first frequency and directing the second ultrasonic energy includes directing the second ultrasonic energy with a peak energy level at a second frequency different than the first frequency.

88. The method of claim 86 wherein directing rust ultrasonic energy includes directing ultrasonic energy having a peak power at a frequency of about 980 kilohertz.

89. A method for treating a flowable substance in a conduit having a first and second end opposite the first end, comprising:
introducing the flowable substance into the conduit through an entrance port toward the second end of the conduit and passing the flowable substance through the conduit along a conduit axis;
directing the ultrasonic energy into the flowable substance from an ultrasonic energy emitter positioned toward the first end of the conduit;
impinging the ultrasonic energy on a parabolically-shaped focusing surface proximate the ultrasonic energy emitter to converge the ultrasonic energy toward a focal point positioned on the conduit axis and spaced apart from the ultrasonic energy emitter;
directing the ultrasonic energy back toward the ultrasonic energy emitter by impinging the ultrasonic energy on a reflecting surface having a tip positioned at least approximately at the focal point and a curved shape extending radially outwardly from the tip;
changing a phase and/or a chemical composition of a selected constituent in the flowable substance by exposing the flowable substance to the ultrasonic energy as it converges toward the focal point and as it passes from the reflecting surface back toward the ultrasonic energy emitter; and
directing the flowable substance through an exit port toward the first end of the conduit.

90. The method of claim 89, further comprising directing a portion of the ultrasonic energy through a first surface of the ultrasonic energy emitter adjacent to the flowable substance by biasing a signal reverser against a second surface of the emitter facing opposite the first surface of the emitter without adhering the signal reverser to the emitter.
